(19) United States
(12) Patent Application Publication
(10) Pub. No.: US 2010/0040704 A1
(43) Pub. Date: Feb. 18, 2010

(54) Composition and method for weight reduction

(76) Inventor: Paul Ling Tai, Chelsea, MI (US)

Correspondence Address: PAUL R. MARTIN, 5333 BAYRIDGE COURT, FAIRFIELD, CA, 94534, US

(21) Appl. No.: 12/228,845

(22) Filed: Aug. 18, 2008

(51) Int. Cl. A61K 36/48; A61P 3/04

(52) U.S. Cl. 424/725

(57) ABSTRACT

Disclosed is a composition and method for facilitating weight loss in individuals. The composition is a physiologically effective amount of the herbal substance Bauhinia in a suitable carrier. The carrier can be water, alcohol, mixtures thereof, capsules, powders, tinctures, liposomes, chewing gum, lozenges, candies, food, skin creams, or lotions. The method is to administer a physiologically effective amount of the herbal substance to an individual seeking to lose weight. The preferred herbal substance is obtained from the leaves of a Bauhinia forficata tree.

### BACKGROUND OF THE INVENTION

(a) Field of the Invention

This invention relates to a composition and method for achieving weight loss or reduction in a human being. More particularly, it relates to a composition containing a herbal substance found in the Amazon forest and elsewhere that is dissolved in or otherwise incorporated into a suitable carrier, and to a method of facilitating weight loss that involves administering a physiologically effective amount of the composition to a person desiring to lose weight.

It is generally known that in the United States of America, 60% of the population is overweight (100 million) or obese (40 million) or morbidly obese (3 million). Among children 77% are overweight and 17% are obese, a 300% increase over 1980. These conditions are associated with numerous medical problems, such as cardiovascular disease, diabetes and various forms of cancer. Effective treatment options are somewhat limited, are expensive and/or encumbered with high risks.

There are many factors that cause human beings to become overweight. Some pathways are set forth below.

### Pathways to Overweight & Obesity

1. Appetite derangement—over eating
2. Abnormal Cortisol metabolism and insulin receptor insensitivity
3. Chronic increased sugar levels in blood
4. Abnormal Leptin activity
   The foregoing conditions are caused by:
1. Dysfunctional Pituitary-Hypothalamus-Adrenal (PHA) a major Master Control Center located in the brain. Increased glandular hyper activity is an Automatic reflex as a result to emotional stress (boss, work, fear, anxiety) or physical stress (sickness, injury, over consumption of food and sugar).
2. Pituitary gland over producing ACTH (adrenal corticotropin hormone) which in turn stimulates the adrenal gland to produce Cortisol *(The Stress of Life by Hans Selye, MD., McGraw Hill Co.) & gluco corticoid. Both of these hormones increase production of sugar in the circulating blood.
3. Overeating, saturated fat, refined sugar (soda, punch, chips, sweets, candy, cake)
4. Leptin in production from fat cells causing food addiction & increasing food consumption.

Increased Chronic Cortisol activity leads to increased circulation of blood sugar resulting in overproduction of insulin to lower the blood sugar which in time causes diminished efficiency or insensitivity of insulin receptors (moves available circulating sugar into the cell) that actually leads to continued elevation (Type II Diabetes) of blood sugar for prolonged periods.

The body reacts to prolonged excess blood glucose by depositing the excess sugar into fat cells as storage for future use, typically in the body mid-section, i.e., the waist area and hips in apple shaped body types and hips and thighs in pear shaped bodies.

Chronic overproduction of cortisol also causes loss of muscle (skinny legs and fat butt and waist) and lower thyroid metabolism by lowering production of active thyroid as as decreased conversion of T4 deiodinase activity to T3. *(C. Tsigos et al. J. Psycho SOM. Res 2002:53:865-71).Typically these individuals consume large quantities of high fats *(J. Knipers, et al. J. Phys. Endo. Met. 2000. 279:1286-93) and processed sugar (pops, sodas, ice cream, chocolate) and proportional larger percentage of diet in high glycemic refined carbohydrate (pasta, potato French fries, breads, chips, breading) These diets exacerbate the vicious cycle and dysfunctional PHA axis.

Lastly, there is a Gut-Brain Axis control where a normal functioning stomach with food will generate a hormone, and signal the brain to stop eating. This “fullness hormone” is called Leptin. This hormone is produced by the fat cells & lining of the stomach. The more one eats, the more it is produced. Higher Leptin levels cause increased eating, a vicious cycle. Leptin activity in the hypothalamus gland establishes a “Set Point” for the body's weight. (Friedman J M. The Function of Leptin in Nutrition, Weight and Physiology. Nutr. Rev. October 2002, 60); (Meister B Control of food intake via Leptin receptors in the hypothalamus. Vitam Horm 2000; 9:265-304.) Leptin causes craving and addiction for food by up regulating increased Dopamine. Higher levels of craving for food, giving a pleasure and reward response to individual. (Krugel U, et al. Eur. J. Pharma Dec. 15, 2003:482,185-7). Increased sugar stimulates excessive production of Leptin, the sweet receptors and taste buds of the tongue enhances behavior to eat more sugar cravings. (Shiglmura N. et al. Endocrinology February 2004 145(2): 839-47) Very much like the mechanism of cocaine, heroin, or sucrose addiction and behavior reinforcing addictive conditioning. (Di Ciano P Neuropharmacology 2004:47)

Scientists of Univ. of College of London have identified yet another set of hormones called PZY I & PZY II. These hormones allow for the slow and fast relaxation of the stomach to accommodate more food. At resting state the stomach hollow is approximately 75 ml (2½ oz). With the help of these hormones, the stomach expands to 25 fold in volume to accept additional food 1.9 liters or 62.5 oz or 4 lbs. The increased consumption of dietary fat (long chain fat) shows activation of inflammatory Adipokines causing further dysfunction of the Gut Brain Axis of communication suppressing normal production of fullness hormone communication with the P.H.A. axis. This is a Leptin caused inflammation (Trayhurn P et al, Br. J. Nutr. September 2004:92(3) 347-55) Increased inflammation (C-Reactive Protein) and visceral adiposity (Saigoy Y. Diab. Obes. Met Jul. 6, 2004(9) 249-58.) The increase in Leptin and CRP is the primary risk and cause of heart attack (Thoagersan A M. Et al., Eur. J. Cardio. Preventive Rehab. Feb. 11, 2004(1) 53-40.)

The search for safe and effective anti-obesity agents is ongoing. It has now been discovered that the use of a composition containing a herbal substance known as Bauhinia is effective in causing weight loss in overweight individuals when administered in physiologically effective amounts.

(b) Description of Related Art

The following references are background material relating to the invention.
1. Hobbs, L. The New Diet Pills. Ch. 7-9. Pragmatic Press, Ca. 2000.
2. Taylor, L. The Healing Power of Rainforest Herbs. Square One Publishers, NJ. 2005.
3. Estrada, O., et al. Evaluation of flavonoids from Bauhinia megalandra leaves as inhibitors of glucose-6-phosphatase system. Phytother. Res. 2005; 19(10): 859-63.
4. Lemus, I., et al. Hypoglycemic activity of four plants used in Chilean popular medicine. Phytother. Res. 1999; 13(2): 91-4.
5. Juliani, C. Hypoglycemic action of bauintrato (Bauhinia forficata preparation) new clinical and experimental study. J. Clin. 1941; 22: 17.
6. Fuentes, O., et al. Hypoglycemic activity of Bauhinia candicans in diabetic induced rabbits. Fitoterapia. September 2004; 75 (6): 527-32.
7. Lino, S., et al. Antidiabetic activity of Bauhinia forficata decoction in alloxan-diabetic rats. Biol. Pharm. Bull. 2004; 27(1): 125-7.
8. de Sousa, E., et al. Hypoglycemic effect and antioxidant potential of kaempferol-4,7-O-(alpha)-dirhamnoside from Bauhinia forficata leaves. J. Nat. Prod. 2004; 67(5): 829-32.
9. Damasceno, D. C., et al. Effect of Bauhinia forficata extract in diabetic pregnant rats: maternal repercussions. Phytomedicine. 2004; 11(2-3): 196201.
10. Pepato, M. T., et al. Evaluation of toxicity after one-months treatment with Bauhinia forficata decoction in streptozotocin-induced diabetic rats. BMC Complement. Altem. Med. Jun. 8, 2004; 4: 7.

### BRIEF SUMMARY OF THE INVENTION

This invention relates to a composition and method for facilitating weight loss in human beings. The composition comprises physiologically effective amounts of the herbal substance Bauhinia in a suitable carrier. The method is a method of facilitating weight loss in human beings which comprises administering a physiologically effective amount of the composition to an individual.

### DETAILED DESCRIPTION OF THE INVENTION

Bauhinia Weight Loss Solution

The weight loss composition of this invention comprises a formulation of extracts of Bauhinia—Saponins, Kempferitrin, flavanoids, astragalin, alkaloids, micro-glycosides, bauhinosides, betasitosterol, flavonols, guanidine, organic acids, quercimocides, rhamnose, and saponins.

Set forth below is a list of the Types & Species of Bauhinia that can be used in the composition and method of this invention.

Bauhinia racemosa

Bauhinia variegata

Bauhinia tarapotensis

Bauhinia divaricata

Bauhinia monandra

Bauhinia pauletia

Bauhinia ungulata

Bauhinia candicans

Bauhinia forficata

Bauhinia grandiflora Juss

Bauhinia purpurea L.

Bauhinia candida Ait.

Bauhinia bariegata L.

Bauhinia macranthera

Bauhinia bartletti

Bauhinia ramosissima

Bauhinia retifolia

Bauhinia cercideae

Bauhinia caesalpinioideae

Bauhinia fabaceae

Bauhinia kalbreyeri

Bauhinia mauca

Bauhinia uruguayensis

Bauhinia splendens

The preferred herbal substance for use in the composition and method of this invention is obtained from leaves of the Bauhinia forficata tree. This tree is found in the Amazon forest of South America, including Ecuador. However, even though it is preferred to use the leaves, the herbal substance can also be obtained from any part of the tree including the root, bark and branches of it.

Extractions: The Bauhinia herb can be used in connection with the following types of carriers:

Water

Oils

Alcohol

Glyceril

CO2

Organic solvent

Dichloromethanol

Other suitable carriers can also be used.

Delivery: The Bauhinia herb can be administered or delivered to the recipient in the following manner:

Liquid/sprays/mist/drinks

Capsules

Powders

Tincture

Liposome

Sublingual

Chewing gums/Lozenges/Candies/food

Transdermals, Skin creams, lotions, etc.

Methods of preparing herbal compositions are found in the book “The Healing Power of Rainforest Herbs” by Leslie Taylor (Square One Publishers, Inc., 2004), incorporated by reference herein. The concentration of Bauhinia herbal substance in the carrier can range from about 0.1% to 99% or more. The exact concentration will depend on the carrier, and method of administering the herbal substance.

A series of tests were run to demonstrate the efficacy of the composition and method of the invention.

### EXAMPLE 1

In these tests, the Bauhinia herbal substance was a mixture of four varieties of Bauhinia in substantially equal amounts. The varieties were: Bauhinia racemosa, Bauhinia tarapotensis, Bauhinia purpurea L., and Bauhinia candicans.

Test Subjects

| Subject A - | Male 60 year old, 5 ft. 11 in. Taken Bauhinia water/alcohol extract 5 | | | |
|---|---|---|---|---|
| | grams leaves in 10 cc concentrate taken 3 times daily, 15 minutes before | | | |
| | meal. No changes in dietary habits, exercise, or medication. Test run for | | | |
| | 6 weeks. | | | |
| | Before wt: | 7158# | After wt: | 147# |
| | Before BMI: | 22 | After BMI: | 20 |
| | Before Waist: | 33 | After Waist: | 29 |

| | |
|---|---|
| Net wt. loss: | 11 lbs. |
| BMI deceased: | 2 |
| Waist measurement decreased: | 4 inches |

| Subject B - | Female 55 year old, 5 ft. 5 in. Taken Bauhinia leaves dried powder 5 | | | |
|---|---|---|---|---|
| | grams 3 times daily before meal. No changes in dietary habits, exercise, | | | |
| | or medication. Test run for 6 weeks. | | | |
| | Before wt: | 174# | After wt: | 168# |
| | Before BMI: | 29 | After BMI: | 28 |
| | Before Waist: | 37 | After Waist: | 36 |

| | |
|---|---|
| Net wt. loss: | 6 lbs. |
| BMI deceased: | 1 |
| Waist measurement decreased: | 1 inch |

| Subject C - | Male 58 year old, 5 ft. 8 in. Taken 10 drops Bauhinia extract concentrate | | | |
|---|---|---|---|---|
| | in a food candy bar, once times daily in middle of the day on full stomach. | | | |
| | No changes in dietary habits, exercise, or medication. Test run for 6 | | | |
| | weeks. | | | |
| | Before wt: | 181# | After wt: | 174# |
| | Before BMI: | 26.7 | After BMI: | 25.7 |
| | Before Waist: | 38.5 | After Waist: | 37 |

| | |
|---|---|
| Net wt. loss: | 7 lbs. |
| BMI deceased: | 1 |
| Waist measurement decreased: | 1.5 inches |

| Subject D - | Female 46 year old, 5 ft. 4 in. Taken Bauhinia water extracted powder | | | |
|---|---|---|---|---|
| | with 1 gram 2 times daily on empty stomach 30 minutes before meals. No | | | |
| | changes in dietary habits, exercise, or medication. Test run for 6 weeks. | | | |
| | Before wt: | 188# | After wt: | 178# |
| | Before BMI: | 32.3 | After BMI: | 30.6 |
| | Before Waist: | 38 | After Waist: | 35.5 |

| | |
|---|---|
| Net wt. loss: | 10 lbs. |
| BMI deceased: | 1.7 |
| Waist measurement decreased: | 2.5 inches |

The foregoing test results indicate that weight loss in human beings can be facilitated by administering physiologically effective amounts of Bauhinia herbal substance to individuals. The Bauhinia in the above tests was administered in the form of powders of dried leaves or extracts. As used herein, the term “physiologically effective amount” refers to that amount of herbal substance which, when administered, is effective to cause loss of weight in an individual.

Another series of tests were conducted to further demonstrate the efficacy of the compositions and method of the invention.

### EXAMPLE 2

In this example, the results of a four week pilot project studying the effect of the herbal substance, Bauhinia forficata, on weight loss in overweight humans in the absence of dietary or exercise intervention is provided.

Methods and Materials

A total of 31 patients (18 male, 13 female) were recruited for the study. They each signed a consent form and were told the study involved an herb used in South America for weight loss and other purposes, and that the substance to be taken was generally regarded as safe. They were instructed to keep their diet and exercise unchanged. Patients were randomly assigned to a treatment or placebo group and instructed to take 5 ml. of the test material (either Bauhinia leaf extract or a placebo solution) 30 minutes before each meal. They were also given a vitamin/mineral supplement to take each morning.

Measurements of weight and circumference of waist, chest, and hips were made and recorded at the start of the study and four weeks. Participants were asked about side effects at each visit. Results were recorded for those who completed the study and analyzed by ANOVA.

TABLE 1
Demographic Data

| | Group A (Placebo) | Group B (Treatment) |
|---|---|---|
| Number | 17 | 14 |
| Gender | Male 11, Female 6 | Male 7, Female 7 |
| Age (M years) | 50.8 | 54.3 |
| Weight (lbs) | 242.41 | 216.36 |
| Height (inches) | 69.14 | 67.96 |
| BMI (mean) | 35.26 | 32.35 |
| Waist (inches) | 44.94 | 42.29 |
| Hip (inches) | 46.97 | 46.21 |
| Chest (inches) | 44.74 | 44.26 |

Results

All subjects completed the study. Table 2 below summarizes the changes in various measurements over the study period treatment for each of the groups. Comparing each group's final to initial values revealed no statistically significant changes, although the differences approached significance for the following parameters in the treatment group: weight (p=0.06), BMI (p=0.06), waist circumference (p=0.06), and waist to hip ratio (0.888). Comparing the amount of change between the two groups revealed that there was a statistically significant difference (p<0.008) in the change in BMI between the two groups. There were no reported adverse effects in either group during the duration of the study. Several subjects reported increased energy or improved sense of well being. One reported “darkening of hair” as a beneficial side effect. The statistically significant effect of the bauhinia extract on weight loss and various measurements did not include dietary or exercise intervention. There was no evidence of toxicity.

TABLE 2
Comparison of Results

| Group A (Placebo) | | | |
|---|---|---|---|
| Group B (Treatment | Initial | Final | (change) |
| N = 33 | | | |
| A | 17 | | |
| B | 14 | | |
| Gender | | | |
| A | Male 11, Female 6 | | |
| B | Male 7, Female 7 | | |
| **Age (years) | | | |
| A | 50.8 | | |
| B | 54.3 | | |
| **Weight (years) | | | |
| A | 242.410 | 242.941 | 0.529 |
| B | 216.360 | 211.696 | −4.661 |
| **BMI | | | |
| A | 35.259 | 35.318 | 0.0588* |
| B | 32.350 | 31.973 | −0.557 |
| **Waist (inches) | | | |
| A | 44.941 | 44.941 | 0.000 |
| B | 42.286 | 40.693 | −1.593 |
| **Hip (inches) | | | |
| A | 46.971 | 47.235 | 0.265 |
| B | 46.214 | 44.893 | −1.324 |
| **Waist-Hip Ratio (WHR) | | | |
| A | 0.953 | 0.947 | −0.006 |
| B | 0.914 | 0.905 | −0.008 |

P Value in (change) = 0.007637
Results listed as means

Bauhinia forficata is a small tree that grows in a small area of the Amazonian plateau in Ecuador. It has long held a place in the folk medicine of South America. Its bark has been used as an anti-diarrheal and was not used in the preparation involved in the present study. The leaves have been used for a variety of purposes, including in the treatment of diabetes, as a general tonic, as an antivenin and as a vermifuge . It has also been associated with beneficial changes in lipid levels indicating there were no toxic effects in either normal or diabetic rats, including pregnant diabetic rats.

Mechanism of Weight Loss

It is not known precisely why the administration of the bauhinia herbal substance is effective to facilitate weight loss in various individuals, however it is postulated that the Bauhinia normalizes PHA-Leptin-Insulin receptor insensitivity and central satiety physiology. Whatever the mechanism, it is clear that it is effective to facilitate weight loss in human beings who take it in physiologically effective amounts.

Although various illustrative embodiments of the composition and method of the invention have been described and shown herein, it is to be understood that the present invention is not limited to the precise embodiments described, and that various other changes and modifications may be affected therein by one skilled in the art without departing from the spirit and scope of the invention. All such changes and modifications are intended to be included within the scope of the invention as defined by the appended claims.

I claim:

1. A composition which facilitates weight loss in individuals which comprises a physiological effective amount of the herbal substance Bauhinia in a suitable carrier.

2. The composition of claim 1 wherein the carrier is selected from the group consisting of water, alcohol, capsules, powders, tinctures, liposomes, chewing gum, lozenges, candies, food, skin creams, or lotions.

3. The composition of claim 1 in which the herbal substance is an extract of the leaves of a Bauhinia forficata tree.

4. The composition of claim 1 wherein the composition is a 5 ml extract of the leaves of a Bauhinia forficata tree dissolved in a physiologically acceptable alcohol.

5. The composition of claim 1 in which the Bauhinia herbal substance comprises a mixture of substances derived from the leaves of Bauhinia racemosa, Bauhinia tarapotensis, Bauhinia purpurea L., and Bauhinia candicans trees.

6. A method of facilitating weight loss in a human being which comprises administering a physiologically effective amount of the herbal substance Bauhinia to said human being.

7. The method of claim 6 wherein said herbal substance is administered in the form of an extract of the leaves of a Bauhinia tree.

8. The method of claim 7 wherein said bauhinia tree is Bauhinia forficata.

9. The method of claim 6 wherein said Bauhinia herbal substance comprises a mixture obtained from the leaves of Bauhinia racemosa, Bauhinia tarapotensis, Bauhinia purpurea L., and Bauhinia candicans trees.

10. The method of claim 7 wherein said extract is contained in or admixed in a carrier selected from the group consisting of water, alcohol, capsules, powders, tinctures, liposomes, chewing gum, lozenges, candies, food, skin creams, or lotions.

11. The method of claim 10 wherein said carrier containing said herbal substance is administered orally.

12. The method of claim 11 wherein said herbal substance is an extract of Bauhinia forficata leaves dissolved in a water/alcohol carrier.
